(19) United States
(12) Patent Application Publication
(10) Pub. No.: US 2013/0095112 A1
(43) Pub. Date: Apr. 18, 2013

(54) NOVEL METHODS FOR TREATING EGFR-ASSOCIATED TUMORS

(71) Applicant: Genmab A/S, Copenhagen (DK)

(72) Inventor: Jens OVERGAARD, Aarhus-C (DK)

(73) Assignee: GENMAB A/S, Copenhagen (DK)

(21) Appl. No.: 13/723,816

(22) Filed: Dec. 21, 2012

Related U.S. Application Data
(63) Continuation of application No. 12/664,258, filed on Aug. 27, 2010.
(60) Provisional application No. 60/936,623, filed on Jun. 21, 2007.

(51) Int. Cl. A61K 39/395

(52) U.S. Cl. CPC A61K 39/3955
USPC 424/142.1; 424/172.1; 424/158.1; 530/389.1; 604/20

(57) ABSTRACT

The invention relates to a method for the treatment of an EGFR-associated tumor comprising administration, to an individual in need thereof, of an anti-EGFR antibody in combination with radiotherapy and a radiosensitising agent. In one embodiment, the treatment further comprises administration of a chemotherapeutic agent.

### RELATED APPLICATIONS

This application is continuation of U.S. application Ser. No. 12/664,258, filed Aug. 27, 2010, which is a 35 U.S.C. 371 national stage filing of International Application No. PCT/DK2008/050142, filed Jun. 17, 2008, which claims priority to U.S. Provisional Application No. 60/936,623, filed on Jun. 21, 2007. The contents of the aforementioned applications are hereby incorporated by reference.

### FIELD OF THE INVENTION

The present invention relates to novel methods for treating cancer associated with the epidermal growth factor receptor (EGFR).

### BACKGROUND OF THE INVENTION

The epidermal growth factor receptor (EGFR) is a tyrosine kinase receptor with critical functions in the regulation of cell proliferation, differentiation and survival (Ullrich and Schlessinger (1990) Cell 61:203-212). Dysregulated function or expression of the EGFR is observed in common cancers such as lung, colon, head and neck, and also in non-epithelial malignancies such as glioblastomas—often correlating with a poor prognosis for the patients. Due to its documented involvement in tumorigenesis, EGFR constitutes a promising molecule for targeted therapy (Mendelsohn (2002) J. Clin. Oncol. 20:1S-13S).

So far, two EGFR-directed approaches have been successfully introduced into clinical practice: small molecule tyrosine kinase inhibitors, and EGFR-directed monoclonal antibodies (Baselga et al. (2005) J. Clin. Oncol. 23:2445-2459). Anti-EGFR antibodies that have been tested in clinical trials include cetuximab (C225), panitumumab (E7.6.3), nimotuzumab (hR3), matuzumab (425), zalutumumab (2F8) and ch806.

Anti-EGFR therapy may be combined with other forms of anti-cancer treatment, such as chemotherapy or radiotherapy. Robert et al. (2001) J. Clin. Oncol. 19:3234-3243 describe a phase I study of anti-epidermal growth factor receptor antibody cetuximab in combination with radiation therapy in patients with advanced head and neck cancer. WO0245653 describes a method of inhibiting growth of tumors that express growth factor receptors, e.g. EGFR, wherein the method comprises administration of an anti-growth factor antibody, a chemotherapeutic agent and radiation therapy. In a study that did not include anti-EGFr antibody therapy, Overgaard et al. (1998) Radiother and Oncology 46:135-146 observed positive effects of co-administration of nimorazole as a hypoxic radiosensitiser of primary radiotherapy in supraglottic larynx and pharynx carcinoma.

While the above-described therapies have proved to be of significant benefit for cancer therapy, a need for further improvement of cancer therapy remains, in particular for aggressive EGFR-associated cancers, e.g. lung and head and neck cancers, which still have a poor prognosis.

### SUMMARY OF THE INVENTION

In a first main aspect, the invention relates to a method for the treatment of an EGFR-associated tumor comprising administration, to an individual in need thereof, of an anti-EGFR antibody in combination with radiotherapy and a radiosensitising agent.

In a further main aspect, the invention relates to an anti-EGFR antibody for use a medicament for the treatment of an EGFR-associated cancer, wherein said anti-EGFR antibody is to be administered in combination with radiotherapy and a radiosensitising agent.

Furthermore, in a main aspect, the invention relates to the use of an anti-EGFR antibody for the preparation of a medicament for the treatment of an EGFR-associated cancer, wherein said anti-EGFR antibody is to be administered in combination with radiotherapy and a radiosensitising agent.

### DETAILED DESCRIPTION OF THE INVENTION

### Definitions

When used herein, the term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H}) and a heavy chain constant region. The heavy chain constant region typically is comprised of three domains, C_{H}1, C_{H}2, and C_{H}3. Each light chain typically is comprised of a light chain variable region (abbreviated herein as V_{L}) and a light chain constant region. The light chain constant region typically is comprised of one domain, C_{L}. The V_{H} and V_{L} regions may be further subdivided into regions of hyper-variability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of V_{H} CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions for significant periods of time such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to modulate a physiological response associated with antibody binding to the antigen and/or time sufficient for the antibody to recruit an Fc-mediated effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation.

As indicated above, the term antibody herein, unless otherwise stated or clearly contradicted by context, includes fragments of an antibody that retain the ability to specifically bind to an antigen. It has been shown that the antigen-binding function of an antibody may be performed by fragments of a full-length antibody. Examples of binding fragments encompassed within the term “antibody” include, but are not limited to: (i) a Fab fragment, a monovalent fragment consisting of the V_{L}, V_{H}, C_{L} and C_{H}1 domains; (ii) F(ab)₂ and F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al; Trends Biotechnol 2003 November; 21(11):484-90); (vi) camelid or nanobodies (Revets et al; Expert Opin Biol Ther. 2005 January; 5(1):111-24), and (vii) an isolated complementarity determining region (CDR). Furthermore, although the two domains of the Fv fragment, V_{L} and V_{H}, are coded for by separate genes, they may be joined, using recombinant methods, by a synthetic linker that enables them to be made as a single protein chain in which the V_{L} and V_{H} regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

Antibodies interact with target antigens primarily through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted into framework sequences from a different antibody with different properties (see for instance Riechmann, L. et al., Nature 332, 323-327 (1998), Jones, P. et al., Nature 321, 522-525 (1986) and Queen, C. et al., PNAS USA 86, 10029-10033 (1989)).

It also should be understood that the term antibody also generally includes polyclonal antibodies, monoclonal antibodies (mAbs), antibody-like polypeptides, such as chimeric antibodies and humanized antibodies, anti-idiotypic (anti-Id) antibodies to antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding, such as amino acid residues which are effectively blocked by the specifically antigen binding peptide (in other words, the amino acid residue is within the footprint of the specifically antigen binding peptide).

As used herein, the term “effector cell” refers to an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, neutrophils, polymorphonuclear cells, granulocytes, mast cells, and basophiles. Some effector cells express specific Fc receptors and carry out specific immune functions. In some embodiments, an effector cell is capable of inducing antibody-dependent cellular cytotoxicity (ADCC), such as a neutrophil capable of inducing ADCC. For example, monocytes, macrophages, which express FcR, are involved in specific killing of target cells and presenting antigens to other components of the immune system, or binding to cells that present antigens. In some embodiments, an effector cell may phagocytose a target antigen, target cell, or microorganism.

The term “human antibody”, as used herein, is intended to include antibodies having variable and constant regions derived from human germline immunoglobulin sequences. The human antibodies of the present invention may include amino acid residues not encoded by human germ line immunoglobulin sequences (for instance mutations introduced by random or site-specific mutagenesis in vitro or by somatic mutation in vivo). However, the term “human antibody”, as used herein, is not intended to include antibodies in which CDR sequences derived from the germ line of another mammalian species, such as a mouse, have been grafted into human framework sequences. As used herein, a human antibody is “derived from” a particular germ line sequence if the antibody is obtained from a system using human immunoglobulin sequences, for instance by immunizing a transgenic mouse carrying human immunoglobulin genes or by screening a human immunoglobulin gene library, and wherein the selected human antibody is at least 90%, such as at least 95%, for instance at least 96%, such as at least 97%, for instance at least 98%, or such as at least 99% identical in amino acid sequence to the amino acid sequence encoded by the germ line immunoglobulin gene. Typically, outside the heavy chain CDR3, a human antibody derived from a particular human germ line sequence will display no more than 10 amino acid differences, such as no more than 5, for instance no more than 4, 3, 2, or 1 amino acid difference from the amino acid sequence encoded by the germ line immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germ line immunoglobulin sequences. The human monoclonal antibodies may be generated by a hybridoma which includes a B cell obtained from a transgenic or transchromosomal nonhuman animal, such as a transgenic mouse, having a genome comprising a human heavy chain transgene and a light chain transgene, fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared there from (described further elsewhere herein), (b) antibodies isolated from a host cell transformed to express the antibody, such as from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germ line immunoglobulin sequences. In certain embodiments, however, such recombinant human antibodies may be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germ line V_{H} and V_{L} sequences, may not naturally exist within the human antibody germ line repertoire in vivo.

As used herein, “antibody capable of binding X” or “antibody which binds” refers to the binding of an antibody to a predetermined antigen X. Typically, the antibody binds with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined by for instance surface plasmon resonance (SPR) technology in a BIAcore 3000 instrument using the antigen as the ligand and the antibody as the analyte, and binds to the predetermined antigen with an affinity corresponding to a K_{D} that is at least ten-fold lower, such as at least 100 fold lower, for instance at least 1000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low, then the amount with which the affinity for the antigen is lower than the affinity for a non-specific antigen may be at least 10,000 fold. Binding affinity also may be determined by equilibrium methods (for instance enzyme-linked immunoabsorbent assay (ELISA) or radioimmunoassay (RIA)).

The term “K_{D}” (M), as used herein, refers to the dissociation equilibrium constant of a particular antibody-antigen interaction.

Antigen binding is preferably specific. The term “specific” herein refers to the ability of an antibody, e.g. an anti-EGFR antibody, to recognize an epitope within an antigen, e.g. EGFR, while only having little or no detectable reactivity with other portions of the antigen or with another, unrelated, antigen. Specificity may be relatively determined by competition assays as described herein. Specificity can more particularly be determined by any of the epitope identification/characterization techniques described herein or their equivalents known in the art. An antibody specific for a particular antigenic determinant may nonetheless cross-react with other biomolecules. For instance, an anti-EGFR antibody that binds human EGFR may cross-react with EGFR homologues from other species.

As used herein, the terms “inhibits binding” and “blocks binding” (for instance when referring to inhibition/blocking of binding of a ligand to EGFR) are used interchangeably herein and encompass both partial and complete inhibition/blocking. The inhibition/blocking of binding of a ligand to a receptor normally reduces or alters the normal level or type of cell signaling that occurs when a ligand binds to the receptor. Inhibition and blocking are also intended to include any measurable decrease in the binding affinity of a ligand to its receptor due to a binding protein, e.g. an antibody. Binding of a ligand to a receptor may e.g. be inhibited by at least about 10%, 20%, 30%, 40%, 50%, 60%, 70%, 80%, 90%, 99%, or 100%.

As used herein, “isotype” refers to the immunoglobulin class (for instance IgG1, IgG2, IgG3, IgG4, IgD, IgA, IgE, or IgM) that is encoded by heavy chain constant region genes.

The terms “transgenic, non-human animal” refers to a non-human animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-EGFR antibodies when immunized with human EGFR antigen and/or cells expressing EGFR. The human heavy chain transgene may be integrated into the chromosomal DNA of the mouse, as is the case for transgenic mice, for instance HuMAb mice, such as HCo7 or HCo12 mice, or the human heavy chain transgene may be maintained extrachromosomally, as is the case for transchromosomal KM mice as described in WO02/43478. Such transgenic and transchromosomal mice (collectively referred to herein as “transgenic mice”) are capable of producing multiple isotypes of human monoclonal antibodies to a given antigen (such as IgG, IgA, IgM, IgD and/or IgE) by undergoing V-D-J recombination and isotype switching. Transgenic, nonhuman animal can also be used for production of antibodies against a specific antigen by introducing genes encoding such specific antibody, for example by operatively linking the genes to a gene which is expressed in the milk of the animal.

The antibodies used in the present invention are typically used in and provided in an at least substantially isolated form. An “isolated” molecule refers to a molecule that is not associated with significant levels (such as more than about 1%, more than about 2%, more than about 3%, or more than about 5%) of any extraneous and undesirable physiological factors, such as non-EGFR binding biomolecules contained within a cell or animal in which the antibody is produced. An isolated molecule also refers to any molecule that has passed through such a stage of purity due to human intervention (whether automatic, manual, or both).

“Treatment” means the administration of an effective amount of a therapeutically active compound of the present invention with the purpose of easing, ameliorating, or eradicating (curing) symptoms or disease states.

When used herein, the term “EGFR-associated tumor” refers to a tumor which has dysregulated function or expression of EGFR. For example, an EGFR-associated tumor may be a tumor which overexpresses EGFR and/or expresses mutant forms of EGFr, such as EGFR variant III.

### Further Aspects and Embodiments of the Invention

In a first main aspect, the invention relates to a method for the treatment of an EGFR-associated tumor comprising administration, to an individual in need thereof, of an anti-EGFR antibody in combination with radiotherapy and a radiosensitising agent.

In a further main aspect, the invention relates to an anti-EGFR antibody for use a medicament for the treatment of an EGFR-associated cancer, wherein said anti-EGFR antibody is to be administered in combination with radiotherapy and a radiosensitising agent.

Furthermore, in a main aspect, the invention relates to the use of an anti-EGFR antibody for the preparation of a medicament for the treatment of an EGFR-associated cancer, wherein said anti-EGFR antibody is to be administered in combination with radiotherapy and a radiosensitising agent.

### Anti-EGFR Antibodies for Use in the Methods of the Invention

The method of the invention comprises the administration of an antibody that binds human EGFR, i.e. an anti-EGFR antibody.

In one embodiment, the anti-EGFR antibody is a human antibody. In another embodiment, the anti-EGFR antibody is a chimeric antibody. In another embodiment, the anti-EGFR antibody is a humanized antibody. In a further embodiment, the anti-EGFR antibody is a polyclonal antibody. In a yet further embodiment, the anti-EGFR antibody is a monoclonal antibody. In an even further embodiment, the anti-EGFR antibody is a human monoclonal antibody.

In a further embodiment, the anti-EGFR antibody is an intact antibody, i.e. a full-length antibody rather than a fragment of an antibody.

An anti-EGFR antibody used in the method of the present invention may have any suitable affinity and/or avidity for one or more epitopes contained in EGFR. Preferably, the antibody used binds to human EGFR with an equilibrium dissociation constant (K_{D}) of at most 10⁻⁸ M, preferably at most 10⁻¹⁰ M.

In one embodiment, the anti-EGFR antibody used is an antibody as described in WO02/100348, WO04/056847, WO200556606, WO05/012479, WO05/10151, U.S. Pat. No. 6,794,494, EP1454917, WO0314159, WO02092771, WO0312072, WO02066058, WO0188138, WO98/50433, WO98/36074, WO96/40210, WO 96/27010, US2002065398, WO95/20045, EP586002, U.S. Pat. No. 5,459,061 or U.S. Pat. No. 4,943,533.

Preferred antibodies for use in the present invention include zalutumumab (2F8, described in WO02/100348 and WO04/056847), cetuximab (Erbitux), nimotuzumab (h-R3), panitumumab (ABX-EGF), and matuzumab (EMD72000), or a variant antibody of any of these, or an antibody which is able to compete with any of these, such as an antibody recognizing the same epitope as any of these. Competition may be determined by any suitable technique. In one embodiment, competition is determined by an ELISA assay. Often competition is marked by a significantly greater relative inhibition than 5%, 10% or 25%, as determined by ELISA analysis.

Thus, in one preferred embodiment, the present invention uses anti-EGFR antibody zalutumumab (2F8) or a variant thereof or an antibody which is able to compete with zalutumumab, such as an antibody, e.g. a human antibody, that binds the same epitope on EGFR as zalutumumab.

Preferred variant antibodies include antibodies comprising the same heavy chain CDR3 region as zalutumumab, i.e. the heavy chain CDR3 sequence set forth in SEQ ID NO:3. Other preferred variant antibodies are antibodies having a variant sequence which is at least 70%, such as at least 80%, e.g. at least 90%, such as at least 95%, e.g. at least 98% or at least 99% identical to the heavy chain variable region of 2F8 and/or at least 70%, such as at least 80%, e.g. at least 90%, such as at least 95%, e.g. at least 98% or at least 99% identical to the light chain variable region of 2F8 (WO 02/100348).

In another embodiment, said anti-EGFR antibody comprises the six CDR sequences set forth in SEQ ID NO:1-6.

Further preferred anti-EGFR antibodies for use in the invention comprise antibodies that have one or more of the following properties:
a) the ability to opsonize a cell expressing EGFR;
b) the ability to inhibit growth and/or mediate phagocytosis and killing of cells expressing EGFR (e.g., a tumor cell) in the presence of human effector cells at a concentration of about 10 μg/ml or less (e.g., in vitro).

Anti-EGFR antibodies used in the present invention may be in any suitable form with respect to multimerization. Also, if desired, the class of anti-EGFR antibody used in the present invention may be switched by known methods. Thus, the effector function of the antibodies of the present invention may be changed by isotype switching to, e.g., an IgG1, IgG2, IgG3, IgG4, IgD, IgA, IgE, or IgM antibody for various therapeutic uses.

In one embodiment, the anti-EGFR antibody used in the present invention is an IgG1 antibody, for instance an IgG1,κ or IgG1,λ isotype. In another embodiment, the anti-EGFR antibody used in the present invention is an IgG3 antibody, for instance an IgG3,κ or IgG3,λ isotype. In yet another embodiment, the antibody used is an IgG4 antibody, for instance an IgG4,κ or IgG4,λ isotype. In a further embodiment, the anti-EGFR antibody used in the present invention is an IgA1 or IgA2 antibody. In an even further embodiment, the anti-EGFR antibody used in the present invention is an IgM antibody. Further possible embodiments of the anti-EGFR antibody used are given below in the section “Production of antibodies”.

### Production of Antibodies

A monoclonal antibody refers to a composition comprising a homogeneous antibody population having a uniform structure and specificity. That an antibody is monoclonal is not to be construed as requiring production of the antibody by any particular method. For example, the monoclonal antibodies used in the present invention may be produced by the hybridoma method first described by Kohler et al., Nature 256, 495 (1975), or may be produced by recombinant DNA methods. Monoclonal antibodies may also be isolated from phage antibody libraries using the techniques described in, for example, Clackson et al., Nature 352, 624-628 (1991) and Marks et al., J. Mol. Biol. 222, 581-597 (1991).

Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc. In one embodiment, human monoclonal antibodies directed against EGFR may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al., Nature 368, 856-859 (1994)). Accordingly, the mice exhibit reduced expression of mouse IgM or κ and in response to immunization, the introduced human heavy and light chain transgenes, undergo class switching and somatic mutation to generate high affinity human IgG,κ monoclonal antibodies (Lonberg, N. et al. (1994), supra; reviewed in Lonberg, N. Handbook of Experimental Pharmacology 113, 49-101 (1994), Lonberg, N. and Huszar, D., Intern. Rev. Immunol. Vol. 13 65-93 (1995) and Harding, F. and Lonberg, N. Ann. N.Y. Acad. Sci 764 536-546 (1995)). The preparation of HuMAb mice is described in detail in Taylor, L. et al., Nucleic Acids Research 20, 6287-6295 (1992), Chen, J. et al., International Immunology 5, 647-656 (1993), Tuaillon et al., J. Immunol. 152, 2912-2920 (1994), Taylor, L. et al., International Immunology 6, 579-591 (1994), Fishwild, D. et al., Nature Biotechnology 14, 845-851 (1996). See also U.S. Pat. No. 5,545,806, U.S. Pat. No. 5,569,825, U.S. Pat. No. 5,625,126, U.S. Pat. No. 5,633,425, U.S. Pat. No. 5,789,650, U.S. Pat. No. 5,877,397, U.S. Pat. No. 5,661,016, U.S. Pat. No. 5,814,318, U.S. Pat. No. 5,874,299, U.S. Pat. No. 5,770,429, U.S. Pat. No. 5,545,807, WO 98/24884, WO 94/25585, WO 93/1227, WO 92/22645, WO 92/03918 and WO 01/09187.

The KM mouse contains a human heavy chain transchromosome and a human kappa light chain transgene. The endogenous mouse heavy and light chain genes also have been disrupted in the KM mice such that immunization of the mice leads to production of human immunoglobulins rather than mouse immunoglobulins. Construction of KM mice and their use to raise human immunoglobulins is described in detail in WO 02/43478. Splenocytes from these transgenic mice may be used to generate hybridomas that secrete human monoclonal antibodies according to well known techniques.

Human monoclonal or polyclonal antibodies used in the present invention, or antibodies used in the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. No. 5,827,690, U.S. Pat. No. 5,756,687, U.S. Pat. No. 5,750,172 and U.S. Pat. No. 5,741,957.

Further, human antibodies used in the present invention or antibodies used in the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (see for instance Hoogenboom et al., J. Mol. Biol. 227, 381 (1991) (phage display), Vaughan et al., Nature Biotech 14, 309 (1996) (phage display), Hanes and Plucthau, PNAS USA 94, 4937-4942 (1997) (ribosomal display), Parmley and Smith, Gene 73, 305-318 (1988) (phage display), Scott TIBS 17, 241-245 (1992), Cwirla et al., PNAS USA 87, 6378-6382 (1990), Russel et al., Nucl. Acids Research 21, 1081-1085 (1993), Hogenboom et al., Immunol. Reviews 130, 43-68 (1992), Chiswell and McCafferty TIBTECH 10, 80-84 (1992), and U.S. Pat. No. 5,733,743). If display technologies are utilized to produce antibodies that are not human, such antibodies may be humanized, for instance as described elsewhere herein.

Anti-EGFR antibodies may be recovered from recombinant combinatorial antibody libraries, such as a scFv phage display library, which may be made with human V_{L} and V_{H} cDNAs prepared from mRNA derived from human lymphocytes. Methods for preparing and screening such libraries are known in the art.

Antibodies used in the present invention may be prepared by recombinant expression in any suitable type of cells or animals. Recombinant antibodies, such as recombinant human antibodies also include antibodies isolated from a recombinant, combinatorial human antibody library, antibodies isolated from an animal, such as a transgenic animal, or antibodies prepared, expressed, created or isolated by any other means that involves splicing of human immunoglobulin-encoding nucleic acid sequences to other nucleic acid sequences exogenous to the human immunoglobulin-encoding nucleic acids and human immunoglobulin-encoding genes. Recombinant human antibodies typically have variable and constant regions derived from human germ line immunoglobulin sequences. In certain embodiments, however, such recombinant human antibodies are subjected to in vitro mutagenesis (or, when an animal transgenic for human Ig sequences is used, in vivo somatic mutagenesis) and, thus, the amino acid sequences of the V_{H} and V_{L} regions of the recombinant antibodies may be sequences that, while derived from and related to human germ line V_{H} and V_{L} sequences, may not naturally exist within the human antibody germ line repertoire in vivo. Suitable methods for antibody production are known in the art and include those described in for instance Harlow et al., Antibodies: A Laboratory Manual, Cold Spring Harbor Laboratory Press, Cold Spring Harbor, N.Y., (1988), Harlow and Lane: Using Antibodies: A Laboratory Manual (Cold Spring Harbor Laboratory Press (1999)), U.S. Pat. No. 4,376,110 and Ausubel et al., eds., Current Protocols In Molecular Biology, Greene Publishing Assoc. and Wiley InterScience N.Y., (1987, 1992). Monoclonal antibodies may be made using the hybridoma method first described by Kohler et al., Nature 256, 495 (1975), or by other well-known, subsequently-developed methods (see, e.g., Goding, Monoclonal Antibodies: Principles and Practice, pp. 59-103 (Academic Press, 1986)). Transformed immortalized B cells may also be used to efficiently produce antibodies used in the present invention. Such cells may be produced by standard techniques, such as transformation with an Epstein Barr Virus, or a transforming gene. (See, e.g., “Continuously Proliferating Human Cell Lines Synthesizing Antibody of Predetermined Specificity,” Zurawaki, V. R. et al., in Monoclonal Antibodies, ed. by Kennett R. H. et al., Plenum Press, N.Y. 1980, pp 19-33.).

Cell lines available as hosts for recombinant protein expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Other cell lines that may be used are insect cell lines, such as Sf9 cells, or bacterial cells or eukaryotic unicellular microorganisms, such as yeast.

Human antibodies of the present invention may also be produced in a host cell transfectoma using, for example, a combination of recombinant DNA techniques and gene transfection methods as is well known in the art, see for instance Morrison, S., Science 229, 1202 (1985).

A “variant” antibody is an antibody that differs from a parent antibody (typically generated by immunization) by one or more suitable amino acid residue alterations, that is substitutions, deletions, insertions, or terminal sequence additions, in the CDRs or other V_{H} and/or V_{L} sequences (provided that at least a substantial amount of the epitope binding characteristics of the parent antibody are retained, if not improved upon, by such changes). Variations in an antibody variant may be made in each of the framework regions, the constant domain, and/or the variable regions (or any one or more CDRs thereof) in a single variant antibody. Alternatively, variations may be made in only one of the framework regions, the variable regions (or single CDR thereof), or the constant domain in an antibody.

A suitable amino acid residue substitution in the context of a CDR variant is any amino acid residue that permits the CDR to interact with the epitope to which the parent CDR is selective/specific and to cooperatively associate with other parent CDRs and/or variant CDRs similarly specific/selective for that epitope. Factors influencing the selection of a suitable amino acid sequence substitution may include the impact of the residue on the conformation of the CDR (e.g., retention of CDR loop structure and flexibility) and the ability to engage in noncovalent interactions (e.g., Van der Waals interactions, hydrogen bonding interactions, ionic interactions, and/or other interactions characteristic of epitope-variable region binding) with the epitope and/or other similar CDRs in a manner similar to or advantageous over the replaced residue in the parent CDR.

The percent identity between two sequences, e.g. variable domain sequences or CDR3 sequences, is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences may be accomplished using a mathematical algorithm, as described in the non-limiting examples below.

The percent identity between two nucleotide sequences may be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences may also be determined using the algorithm of E. Meyers and W. Miller, Comput. Appl. Biosci 4, 11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, about 95% or more (e.g., about 65-99%) of the substitutions in the variant are conservative amino acid residue replacements. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp and Glu |
| Basic Residues | Lys, Arg, and His |
| Hydrophilic Uncharged Residues | Ser, Thr, Asn, and Gln |
| Aliphatic Uncharged Residues | Gly, Ala, Val, Leu, and Ile |
| Non-polar Uncharged Residues | Cys, Met, and Pro |
| Aromatic Residues | Phe, Tyr, and Trp |

Variant anti-EGFR antibodies used in the present invention may comprise framework (FR) alterations, that is outside the hypervariable region, for instance in the Fc region, which alterations may be associated with advantageous properties, such as changing the functional or pharmacokinetic properties of the antibodies. For example, a substitution or other modification (insertion, deletion, terminal sequence additions or combination of any thereof) in a framework region or constant domain may be associated with an increase in the half-life of the variant antibody with respect to the parent antibody, or may be made to alter the immunogenicity of the variant antibody with respect to the parent antibody, to provide a site for covalent or non-covalent binding to another molecule, or to alter such properties as complement fixation, for instance resulting in a decrease or increase of C1q binding and CDC or of FcγR binding and antibody-dependent cellular cytotoxicity (ADCC).

The present invention may also use fragments of antibodies (including variant antibodies). Examples of such antibody fragments include Fab, Fab′, F(ab′)₂, and Fv fragments. Thus, although the discussion herein may focus on antibodies, it should be understood that the embodiments and features of the antibodies may equally be applied to antibody fragments, such as Fab fragments, Fab′ fragments, and scFv peptides, antibody-like peptides (peptides comprising a CDR), and bi- and multi-specific antibodies as appropriate, provided that the molecule retains at least a substantial proportion of the antigen-binding properties of the corresponding complete antibody. In some instances, antibody fragments may be associated with lower antigen-binding affinity, but may offer other advantageous features that may offset for any such loss in affinity.

Antibodies used in the present invention also include antibody derivatives. Such derivatives may be produced by chemically conjugating a radioisotope, protein, or other agent/moiety/compound to the N-terminal side or C-terminal side of the antibody or subunit thereof, an appropriate substituent group or side chain or to a sugar chain in the antibody (see, e.g., Antibody Engineering Handbook, edited by Osamu Kanemitsu, published by Chijin Shokan (1994)). Derivatives may also be generated by conjugation at internal residues or sugars, where appropriate.

In one embodiment, the present invention uses an anti-EGFR antibody that is conjugated to a second molecule that is selected from a radionuclide, an enzyme, an enzyme substrate, and a cofactor. In one embodiment, an anti-EGFR antibody may be conjugated to one or more antibody fragments, nucleic acids (oligonucleotides), nucleases, hormones, immunomodulators, chelators, boron compounds, and the like. These and other suitable agents may be coupled either directly or indirectly to an anti-EGFR antibody.

In one embodiment, anti-EGFR antibody derivatives comprising one or more radiolabeled amino acids are used. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. No. 4,681,581, U.S. Pat. No. 4,735,210, U.S. Pat. No. 5,101,827, U.S. Pat. No. 5,102,990 (U.S. Pat. No. RE35,500), U.S. Pat. No. 5,648,471 and U.S. Pat. No. 5,697,902.

In one embodiment, the present invention uses molecules comprising an anti-EGFR antibody, such as a human anti-EGFR antibody, conjugated to a therapeutic moiety, such as a cytotoxin, a chemotherapeutic drug, an immunosuppressant, or a radioisotope. Such conjugates are referred to herein as “immunoconjugates”. Immunoconjugates which include one or more cytotoxins are referred to as “immunotoxins”.

A cytotoxin or cytotoxic agent includes any agent that is detrimental to (e.g., kills) cells. For a description of these classes of drugs which are well known in the art, and their mechanisms of action, see Goodman et al., Goodman and Gilman's The Pharmacological Basis Of Therapeutics, 8th Ed., Macmillan Publishing Co., 1990. Additional techniques relevant to the preparation of antibody immunotoxins are provided in for instance Vitetta, Immunol. Today 14, 252 (1993) and U.S. Pat. No. 5,194,594.

Suitable therapeutic agents for forming immunoconjugates of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, calicheamicins, duocarmycins, alpha-sarcin, Aleurites fordii proteins, dianthin proteins, Phytolacca americana proteins (PAPI, PAPII, and PAP-S), momordica charantia inhibitor, curcin, crotin, saponaria officinalis inhibitor, gelonin, mitogellin, restrictocin, phenomycin, and enomycin toxins. Therapeutic agents, which may be administered in combination with an anti-EGFR antibody as described elsewhere herein, may also be candidates for therapeutic moieties useful for conjugation to an anti-EGFR antibody. Techniques for conjugating such therapeutic moieties to antibodies, are well known.

### Radiotherapy and Radiosensitising Agents

In addition to anti-EGFR antibody treatment, the method of the invention comprises treatment with a combination of radiotherapy and a radiosensitising agent.

Radiotherapy may comprise conventional radiotherapy or fractionated radiotherapy, such as hyperfractionated or accelerated fractionation radiotherapy. Suitable radiotherapy protocols for use in the method of the invention for the treatment of head and neck cancer have been described in Robert et al. (2001) J. Clin. Oncol. 19:3234-3243 and Overgaard et al. (1998) Radiother and Oncology 46:135-146.

In one embodiment, a total dose of between 40 and 80 Gy, e.g. a total dose of between 60 and 80 Gy, such as a total dose of between 68 and 70 Gy is given, for example in a dose of 5-6 fractions of 1.5-2 Gy per week.

The radiosensitising agent used in the method of the invention may be any agent that sensitises tumor cells to radiotherapy.

In one embodiment, said radiosensitising agent is a hypoxia (also termed “hypoxic”) modifier. In a further embodiment, said radiosensitising agent is selected from the group consisting of: nimorazole, SR-2514, misonidazole, fluosol and tirapazamine.

In an even further embodiment, said radiosensitising agent is nimorazole and said nimorazole is administered in a dosage of between 500 and 4000 mg/m² daily, such as between 750 and 2500 mg/m² daily, e.g. between 1000 and 1500 mg/m² daily.

The radiosensitising agent is usually administered on the same day as the radiation. In one embodiment of the method of the invention, the radiosensitising agent is administered between 10 and 300 minutes before radiotherapy, such as between 60 and 120 minutes before radiotherapy.

### EGFR-Associated Tumors

As explained above, the method of the invention is directed to the treatment of a tumor. In one embodiment, said tumor is selected from the group consisting of: breast tumor, bladder tumor, uterine/cervical tumor, esophageal tumor, pancreatic tumor, colon cancer, colorectal tumor, kidney tumor, ovarian tumor, prostate tumor, head and neck tumor, non-small cell lung tumor and stomach tumor.

In an important embodiment, said tumor is a squamous cell carcinoma of the head and neck (SCCHN).

In an even further embodiment, embodiment, the tumor is selected from glioblastoma, including glioblastoma multiforme astrocytoma, including childhood astrocytoma, glioma, neuroblastoma, neuroendocrine tumors of the gastrointestinal tract, bronchoalveolar carcinoma, follicular dendritic cell sarcoma, salivary gland carcinoma, ameloblastoma, malignant peripheral nerve sheet tumor, endocrine pancreatic tumors, testicular germ cell tumors, including seminoma, embryonal carcinoma, yolk sac tumor, teratoma and choriocarcinoma.

### Dosage Regimens

In the method and use of the invention, the anti-EGFR antibody is given in an effective amount, i.e. in an amount effective, at dosages and for periods of time necessary, in combination with the other therapies, to achieve a desired result.

A therapeutically effective amount may vary according to factors such as the disease state, age, sex, and weight of the individual, and the ability of the agent to elicit a desired response in the individual.

An effective amount for tumor therapy may also be measured by its ability to stabilize the progression of disease. The ability of a compound to inhibit cancer may be evaluated in an animal model system predictive of efficacy in human tumors. Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

As non-limiting examples, treatment according to the present invention may be provided as a daily dosage of the antibody in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In one embodiment, the dosage regimen of said anti-EGFR antibody comprises administration, at least once per 14 days, of a dose of antibody of from 0.1 mg/kg to 500 mg/kg, such as from 0.25 mg/kg to 200 mg/kg, e.g. from 0.5 mg/kg to 200 mg/kg, such as from 1 mg/kg to 100 mg/kg, e.g. from 2 mg/kg to 100 mg/kg, such as from 4 mg/kg to 50 mg/kg, e.g. from 8 mg/kg to 50 mg/kg. In one embodiment, said administration is at least once per week.

In another embodiment of the invention, the anti-EGFR agent is an anti-EGFR antibody and the dosage regimen of the anti-EGFR antibody comprises administration, at least once per 14 days, of a dose of antibody of at least 5 mg, such as at least 10 mg, e.g. at least 25 mg, such as at least 50 mg, e.g. at least 75 mg, such as at least 100 mg, e.g. at least 150 mg, such as at least 200 mg, e.g. at least 250 mg, such as at least 300 mg e.g. at least 350 mg, such as at least 400 mg, e.g. at least 500 mg, such as at least 750 mg, e.g. at least 1000 mg, such as at least 1250 mg, e.g. at least 1500 mg, such as at least 2000 mg. More preferably, the administration of the anti-EGFR antibody is at least once per week.

In a further embodiment, the total duration of the anti-EGFR treatment is at least one month, such as at least two months, e.g. at least four months, such as at least six months.

In some embodiments of the invention, the method of treatment is repeated after an interval of two months or more, such as three months or more, e.g. after six months or more.

### Formulation, Additives and Mode-of-Administration

The pharmaceutical compositions and agents used in the method of the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutically acceptable carriers or diluents as well as any other known adjuvants and excipients should be suitable for the chosen composition of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact (10% or less relative inhibition, 5% or less relative inhibition, etc.) on antigen binding.

A pharmaceutical composition of the present invention may also include diluents, fillers, salts, buffers, detergents (e. g., a nonionic detergent, such as Tween-80), stabilizers, stabilizers (e. g., sugars or protein-free amino acids), preservatives, tissue fixatives, solubilizers, and/or other materials suitable for inclusion in a pharmaceutical composition.

The actual dosage levels of the active ingredients in the pharmaceutical compositions used in the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions may be administered by any suitable route and mode. Suitable routes of administering a composition in vivo and in vitro are well known in the art and may be selected by those of ordinary skill in the art. The antibody used in the invention is preferably administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In a preferred embodiment, the pharmaceutical composition comprising the anti-EGFR antibody is administered by intravenous or subcutaneous injection or infusion.

Regardless of the route of administration selected, the compositions of the present invention, which may be used in the form of a pharmaceutically acceptable salt or in a suitable hydrated form, are formulated into pharmaceutically acceptable dosage forms by conventional methods known to those of skill in the art. A “pharmaceutically acceptable salt” refers to a salt that retains the desired biological activity of the parent compound and does not impart any undesired toxicological effects (see for instance Berge, S. M. et al., J. Pharm. Sci. 66, 1-19 (1977)). Pharmaceutically acceptable carriers include any and all suitable solvents, dispersion media, coatings, antibacterial and antifungal agents, isotonicity agents, antioxidants and absorption delaying agents, and the like that are physiologically compatible with a compound used in the present invention.

Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts. Proper fluidity may be maintained, for example, by the use of coating materials, such as lecithin, by the maintenance of the required particle size in the case of dispersions, and by the use of surfactants.

Pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Pharmaceutical compositions used in the present invention may also comprise isotonicity agents, such as sugars, polyalcohols such as mannitol, sorbitol, glycerol or sodium chloride in the compositions.

Pharmaceutically acceptable diluents include saline and aqueous buffer solutions. The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. Compounds used in the present invention may for instance be admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol.

Prevention of presence of microorganisms may be ensured both by sterilization procedures and by the inclusion of various antibacterial and antifungal agents, for example, paraben, chlorobutanol, phenol, sorbic acid, and the like. In addition, prolonged absorption of the injectable pharmaceutical form may be brought about by the inclusion of agents which delay absorption such as aluminum monostearate and gelatin.

Pharmaceutical compositions of the present invention may also include a suitable salt therefore. Any suitable salt, such as an alkaline earth metal salt in any suitable form (e.g., a buffer salt), may be used in the stabilization of the compound used in the present invention. Suitable salts typically include sodium chloride, sodium succinate, sodium sulfate, potassium chloride, magnesium chloride, magnesium sulfate, and calcium chloride. In one embodiment, an aluminum salt is used.

### Combination with Other Forms of Therapy

In one embodiment, the method of the invention further comprises administration of a chemotherapeutic agent. Thus, this embodiment comprises a method for the treatment of an EGFR-associated tumor comprising administration, to an individual in need thereof, of an anti-EGFR antibody in combination with radiotherapy, a radiosensitising agent and a chemotherapeutic agent.

In one such embodiment, said chemotherapeutic agent is a platinum complex, such as cisplatin or carboplatin.

In a further embodiment, said chemotherapeutic agent, e.g. cisplatin, is administered weekly.

In a preferred embodiment, said chemotherapeutic agent is cisplatin and said cisplatin is administered in at least 5 weekly series of a dosage of from 20 to 60 mg/m², e.g. at least 5 weekly series of a dosage of 40 mg/m² or in at least 3 biweekly series of a dosage of from 50 to 250 mg/m², e.g. at least 3 biweekly series of a dosage of 100 mg/m².

Further suitable dosage regimens for combination of platinum complexes with anti-EGFR antibody therapy include those described in Baselga et al. (2005) J Clin Oncol 23(24):5568-7 and Pfister et al. (2006) J Clin Oncol 24:1072-1078.

In a further embodiment, the method comprises administration of one or more further chemotherapeutics agents, e.g. one or more chemotherapeutic agents selected from the group consisting of: nitrogen mustards, aziridines, alkyl sulfonates, nitrosoureas, non-classical alkylating agents, folate analogs, purine analogs, adenosine analogs, pyrimidine analogs, substituted ureas, antitumor antibiotics, epipodophyllotoxins, microtubule agents, camptothecin analogs, enzymes, cytokines, monoclonal antibodies, recombinant toxins and immunotoxins, cancer gene therapies and cancer vaccines.

In addition to or as alternative to a chemotherapeutic agent, the method of the invention may comprise other further forms of therapy. For example, in one embodiment, the method of the invention comprises administration of one or more further therapies selected from immunosuppressive agents, anti-inflammatory agents, anti-psoriasis agents, radiation therapy, hyperthermia, transplantation, surgery, sunlight therapy, and phototherapy.

In one embodiment, the tumor treated is a head and neck tumor and the method of the invention further comprises elective neck dissection.

In an even further embodiment, the method comprises administration of one or more further therapies selected from the group consisting of immunosuppressive antibodies against MHC, CD2, CD3, CD4, CD7, CD28, B7, CD40, CD45, IFN-gamma, TNF-alpha, IL-4, IL-5, IL-6R, IL-7, IL-8, IL-10, CD11a, CD20, CD58, and soluble IL-15R.

In a yet further embodiment, the method comprises administration of one or more further therapies selected from the group consisting of cyclosporine, azathioprine, mycophenolic acid, mycophenolate mofetil, corticosteroids, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, aspirin, other salicylates, steroidal drugs, nonsteroidal anti-inflammatory drugs, Cox-2 inhibitors, coal tar, vitamin A, anthralin, calcipotrien, tarazotene, corticosteroids, methotrexate, retinoids, etanercept, alefacept, efaluzimab, 6-thioguanine, mycophenolate mofetil, tacrolimus (FK-506) and hydroxyurea.

The present invention is further illustrated by the following examples which should not be construed as further limiting.

### EXAMPLES

### Example 1

Patients diagnosed with advanced (stage III-IV) squamous cell carcinoma of the head and neck are being treated according to the following dosage regimen:
8 mg/kg zalutumumab on each of day 1, 7, 14, 21, 28, 35 and 42,
40 mg/m² cisplatin on each of day 7, 14, 21, 28 and 35, and
2 Gy radiotherapy and 1.2 g/m² nimorazole on each of day 7, 8, 9, 10, 11, 12, 14, 15, 16, 17, 18, 19, 21, 22, 23, 24, 25, 26, 28, 29, 30, 31, 32, 33, 35, 36, 37, 38, 39, 40, 42, 43, 44 and 45, wherein nimorazole is given orally 90 min prior to radiotherapy treatment.

### Example 2

Patients diagnosed with advanced (stage III-IV) squamous cell carcinoma of the head and neck are being treated according to the following dosage regimen:
8 mg/kg zalutumumab on each of day 1, 7, 14, 21, 28, 35 and 42,
100 mg/m² cisplatin on each of day 7, 28 and 49, and
2 Gy radiotherapy and 1.2 g/m² nimorazole on each of day 7, 8, 9, 10, 11, 12, 14, 15, 16, 17, 18, 19, 21, 22, 23, 24, 25, 26, 28, 29, 30, 31, 32, 33, 35, 36, 37, 38, 39, 40, 42, 43, 44 and 45, wherein nimorazole is given orally 90 min prior to radiotherapy treatment.

SEQUENCE LISTING
SEQ ID NO: 1 the heavy chain CDR1
sequence of zalutumumab:
TYGMH
SEQ ID NO: 2 the heavy chain CDR2
sequence of zalutumumab:
VIWDDGSYKYYGDSVKG
SEQ ID NO: 3 the heavy chain CDR3
sequence of zalutumumab:
DGITMVRGVMKDYFDY
SEQ ID NO: 4 the light chain CDR1
sequence of zalutumumab:
RASQDISSALV
SEQ ID NO: 5 the light chain CDR2
sequence of zalutumumab:
DASSLES
SEQ ID NO: 6 the light chain CDR3
sequence of zalutumumab:
QQFNSYPLT

We claim:

1. A method for the treatment of an EGFR-associated tumor comprising administration, to an individual in need thereof, of an anti-EGFR antibody in combination with radiotherapy and a radiosensitizing agent.

2. The method of claim 1, further comprising administration of a chemotherapeutic agent.

3. The method of claim 1, wherein said anti-EGFR antibody binds the same epitope on EGFR as zalutumumab.

4. The method of claim 1, wherein said anti-EGFR antibody is a human antibody.

5. The method of claim 1, wherein said antibody binds to human EGFR with an equilibrium dissociation constant (KD) of at most 10⁻⁸ M, preferably at most 10⁻¹⁰ M.

6. The method of claim 1, wherein said anti-EGFR antibody comprises a heavy chain CDR3 region as set forth in SEQ ID NO: 3.

7. The method of claim 1, wherein said anti-EGFR antibody comprises the six CDR sequences set forth in SEQ ID NO: 1-6.

8. The method of claim 1, wherein said anti-EGFR antibody is zalutumumab.

9. The method of claim 1, wherein the dosage regimen of said anti-EGFR antibody comprises administration, at least once per 14 days, of a dose of antibody of from 0.1 mg/kg to 500 mg/kg, such as from 0.25 mg/kg to 200 mg/kg, e.g. from 0.5 mg/kg to 200 mg/kg, such as from 1 mg/kg to 100 mg/kg, e.g. from 2 mg/kg to 100 mg/kg, such as from 4 mg/kg to 50 mg/kg, e.g. from 8 mg/kg to 50 mg/kg.

10. The method of claim 9, wherein said administration is at least once per week.

11. The method of claim 1, wherein said radiotherapy comprises accelerated fractionation.

12. The method of claim 11, wherein a total dose of between 40 and 80 Gy, e.g. a total dose of between 60 and 80 Gy, such as a total dose of between 68 and 70 Gy is given, for example in a dose of 5-6 fractions of 1.5-2 Gy per week.

13. The method of claim 1, wherein said radiosensitizing agent is a hypoxia modifier.

14. The method of claim 1, wherein said radiosensitizing agent is selected from the group consisting of: nimorazole, SR-2514, misonidazole, fluosol and tirapazamine.

15. The method of claim 1, wherein said radiosensitizing agent is nimorazole and said nimorazole is administered in a dosage of between 500 and 4000 mg/m² daily, such as between 750 and 2500 mg/m² daily, e.g. between 1000 and 1500 mg/m² daily.

16. The method of claim 1, wherein the radiosensitizing agent is administered between 10 and 300 minutes before radiotherapy, such as between 60 and 120 minutes before radiotherapy.

17. The method of claim 2, wherein said chemotherapeutic agent is administered weekly.

18. The method of claim 2, wherein said chemotherapeutic agent is a platinum complex, such as cisplatin or carboplatin.

19. The method of claim 18, wherein said chemotherapeutic agent is cisplatin and said cisplatin is administered in at least 5 weekly series of a dosage of from 20 to 60 mg/m², e.g. at least 5 weekly series of a dosage of 40 mg/m² or in at least 3 biweekly series of a dosage of from 50 to 250 mg/m², e.g. at least 3 biweekly series of a dosage of 100 mg/m².

20. The method of claim 1, wherein said tumor is selected from the group consisting of: breast tumor, bladder tumor, uterine/cervical tumor, esophageal tumor, pancreatic tumor, colorectal tumor, kidney tumor, ovarian tumor, prostate tumor, head and neck tumor, non-small cell lung tumor and stomach tumor.

21. The method of claim 1, wherein said tumor is a squamous cell carcinoma of the head and neck (SCCHN).

22. The method of claim 1, wherein said tumor is selected from glioblastoma, including glioblastoma multiforme astrocytoma, including childhood astrocytoma, glioma, neuroblastoma, neuroendocrine tumors of the gastrointestinal tract, bronchoalveolar carcinoma, follicular dendritic cell sarcoma, salivary gland carcinoma, ameloblastoma, malignant peripheral nerve sheet tumor, endocrine pancreatic tumors, testicular germ cell tumors, including seminoma, embryonal carcinoma, yolk sac tumor, teratoma and choriocarcinoma.

23. The method of claim 1, comprising administration of one or more further therapies selected from immunosuppressive agents, anti-inflammatory agents, anti-psoriasis agents, hyperthermia, transplantation, surgery, sunlight therapy and phototherapy.

24. The method of claim 23, wherein said method further comprises elective neck dissection.

25. The method of claim 2, comprising administration of one or more further chemotherapeutics agents, e.g. one or more chemotherapeutic agents selected from the group consisting of: nitrogen mustards, aziridines, alkyl sulfonates, nitrosoureas, non-classical alkylating agents, folate analogs, purine analogs, adenosine analogs, pyrimidine analogs, substituted ureas, antitumor antibiotics, epipodophyllotoxins, microtubule agents, camptothecin analogs, enzymes, cytokines, monoclonal antibodies, recombinant toxins and immunotoxins, cancer gene therapies and cancer vaccines.

26. The method of claim 1, comprising administration of one or more further therapies selected from the group consisting of immunosuppressive antibodies against MHC, CD2, CD3, CD4, CD7, CD28, B7, CD40, CD45, IFN-gamma, TNF-alpha, IL-4, IL-5, IL-6R, IL-7, IL-8, IL-10, CDIIa, CD20, CD58, soluble IL-15R.

27. The method of claim 1, comprising administration of one or more further therapies selected from the group consisting of cyclosporine, azathioprine, mycophenolic acid, mycophenolate mofetil, corticosteroids, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, aspirin, other salicylates, steroidal drugs, nonsteroidal anti-inflammatory drugs, Cox-2 inhibitors, coal tar, vitamin A, anthralin, calcipotrien, tarazotene, corticosteroids, methotrexate, retinoids, etanercept, alefacept, efaluzimab, 6-thioguanine, mycophenolate mofetil, tacrolimus (FK-506) and hydroxyurea.

28. An anti-EGFR antibody for use a medicament for the treatment of an EGFR-associated cancer, wherein said anti-EGFR antibody is to be administered in combination with radiotherapy and a radiosensitizing agent.

29. The anti-EGFR antibody of claim 28, further comprising administration of a chemotherapeutic agent.

30. Use of an anti-EGFR antibody for the preparation of a medicament for the treatment of an EGFR-associated cancer, wherein said anti-EGFR antibody is to be administered in combination with radiotherapy and a radiosensitizing agent.

31. The use of claim 30, further comprising administration of a chemotherapeutic agent.


SEQUENCE LISTING (6 sequences)

SEQ ID NO: 1 - protein, 5 aa, Homo sapiens
TYGMH

SEQ ID NO: 2 - protein, 17 aa, homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT